(19) United States
(12) Patent Application Publication
(10) Pub. No.: US 2013/0092058 A1
(43) Pub. Date: Apr. 18, 2013

(54) PIVOTAL MEDICAL TRAY

(71) Applicant: Angelita Slagle, Fort Worth, TX (US)

(72) Inventor: Angelita Slagle, Fort Worth, TX (US)

(21) Appl. No.: 13/275,277

(22) Filed: Oct. 17, 2011

(51) Int. Cl. A61G 13/10; A61B 19/02

(52) U.S. Cl. 108/6

(57) ABSTRACT

The present invention is directed to a medical tray system for attachment to a side rail of a surgical table which enables fine control and placement of the medical tray relative to the patient. The pivotal medical tray comprises a base and a shaft having a first end and a second end. The first end of the shaft is joined to the base. A bracket comprising atop edge joined to a bottom section. The shaft extends upwardly and is fastened to the bottom section of the bracket proximate the second end of the shaft, defining a pivot point. The bracket further comprises an arcuate slot in the bottom section. The shaft further comprises a guide extending through said arcuate slot. A tray assembly is joined to the top edge of the bracket such that the bracket and the tray assembly cooperatively and selectively rotate about a pivot axis.

### BACKGROUND

1. Field of the Invention

The present invention relates to a medical tray, more specifically to a medical tray for use during medical procedures.

2. Description of the Related Art

During some surgeries or other medical procedures, a patient is placed on a surgical table. It is often necessary to have a variety of medical instruments or supplies proximate the surgical table readily available for use during the procedure. Prior to the procedure, the medical instruments are sterilized and placed on a sterile medical tray so that they are available to the medical staff during the procedure. Currently, a Mayo stand is the typical medical tray used and moved near the surgical table. It comprises a wheeled base joined to an upwardly extending shaft which is in turn joined to a tray. The typical surgical table includes a flat resting surface for the patient, optionally comprising a plurality of interconnected articulated sections for elevating different parts of a patient's body. A side rail spans at least a portion of the length of each side of the surgical table. Each side rail further optionally includes vertically oriented slots adapted for receipt of attachments.

During the typical medical procedure, the patient is placed on the surgical table. The Mayo stand is wheeled next to the surgical table, with the tray being placed as close as practical to the surgical site, both vertically and horizontally from the patient. As the medical procedure commences, the medical staff pass the instruments from the tray to each other. Often, the medical staff move around the surgical table as the procedure progresses. Being nonintegral with the surgical table, the Mayo stand is deficient in that its base covers some of the area around the surgical table, impeding the medical staff's movement around the patient.

The surgical tray of U.S. Pat. No. 6,471,167 to Myers et al. discloses a tray assembly for attachment to a side rail of a surgical table. It addresses the issue of covering some of the area surrounding the surgical table, but it fails to provide fine control of the vertical height from the patient during the medical procedure. It also fails to provide fine angular control of the tray relative to the patient in response to adjusting the articulated sections of the surgical table or otherwise placing the patient in different positions during the medical procedure.

The deficiencies in current medical trays have been exacerbated by the recent adoption of robot assisted medical procedures. In such procedures, at least one robotic arm having an extension is employed. The extension is guided to the patient at or proximate the surgical site, frequently from acute angles relative to the patient, requiring a clearance zone above the patient as the extension approaches the patient and as the arm is moved during the procedure. This type of procedure also highlights the need for fine placement and control of the medical tray during the procedure. The medical staff seek selective control and placement such that the medical tray can be placed within millimeters to centimeters above the patient in the various possible positions of the patient on the surgical table and robotic arm travel over the patient. In other words, the medical tray should be selectively configurable to provide fine pivotal control and placement when the patient is flat on the surgical table or the surgical table is manipulated to elevate a portion of the patient's body.

For the above reasons, there is a need for a medical tray device which provides fine pivotal control and placement for use with surgical tables during medical procedures.

### SUMMARY

The present invention is directed to a medical tray system for attachment to a side rail of a surgical table. The pivotal medical tray comprises a base and a shaft having a first end and a second end. The proximal end of the shaft is joined to the base. A bracket comprising an upper section joined to a folded section which is in turn joined to a bottom section. The shaft extends upwardly and is fastened to the bottom section of the bracket, proximate the distal end of the shaft, defining a pivot point. The bracket further comprises an arcuate slot in its bottom section. The shaft further comprises a guide extending through the arcuate slot. A tray assembly is joined to the upper section of the bracket such that the bracket and the tray assembly cooperatively and selectively rotate about a pivot axis.

These and other features, aspects, and advantages of the invention will become better understood with reference to the following description, appended claims, and accompanying drawings.

### BRIEF DESCRIPTION OF THE DRAWINGS

FIG. 1 depicts a top perspective view of an embodiment of the invention as it may appear in a medical environment;

FIG. 2 depicts an exploded side perspective view of the embodiment of FIG. 1;

FIG. 3 depicts a bottom perspective view of the embodiment of FIG. 1;

FIG. 4 depicts an alternate bottom perspective view of the embodiment of FIG. 1; and

FIG. 5 depicts a side perspective view of the embodiment of FIG. 1.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure, or manner.

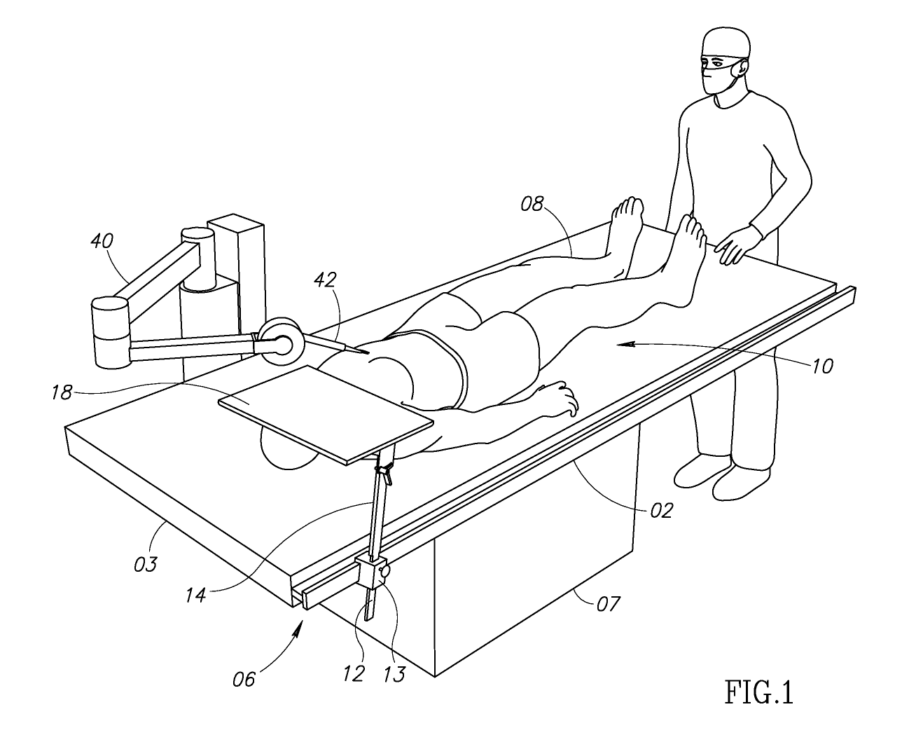

The current invention provides a pivotal medical tray 10 for attachment to a surgical table 06. Referring to FIG. 1, an embodiment of the invention 10 as it may exist in the medical environment is illustrated. Illustrated are the surgical table 06 and the pivotal medical tray 10. The surgical table 06 is comprised of a slab 03, a support 07, and at least one side rail assembly 04. The slab 03 has an upper surface presenting a resting surface for a patient 08. The slab 03 may be further comprised of interconnected, articulating sections such that the patient's 08 head, mid-section, or lower body can be elevated. The side rail assembly 04 is comprised of a side rail 02 and at least one mount. The side rail 02 is mounted to the side of the slab 03. The side rail 02 optionally has vertically oriented slots for receipt of attachments. The slab 03 rests on the support 07.

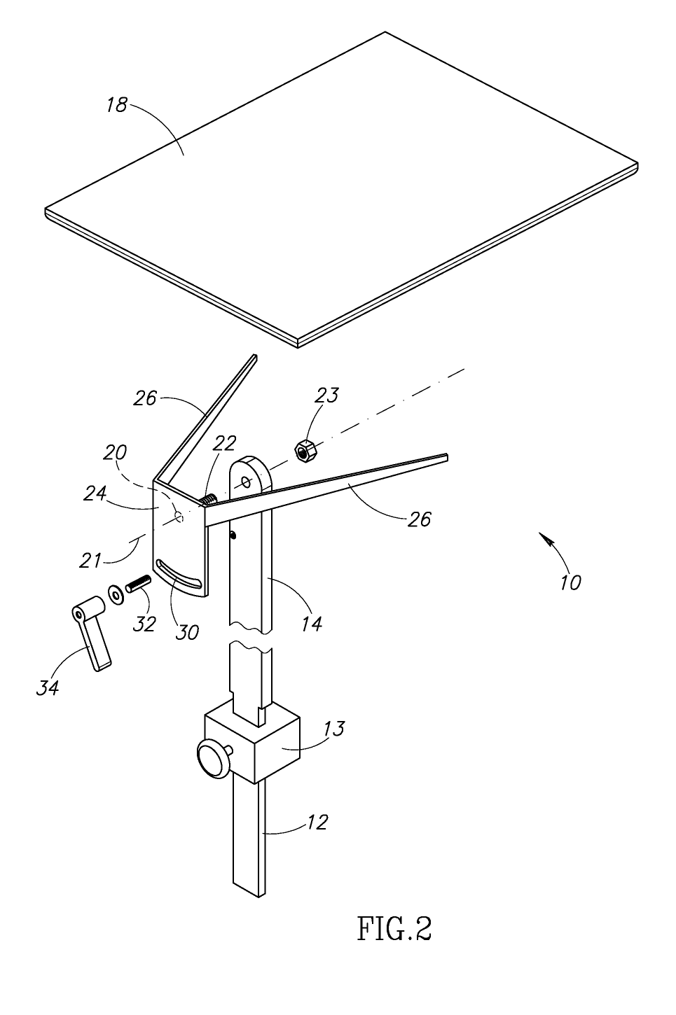

Referring to FIG. 2, the pivotal medical tray 10 is comprised of a base 12, a shaft 14, a bracket 24, and a tray assembly 16. The base 12 is a rigid segment of material extending upwardly, operable to removably attach the pivotal medical tray 10 to the side rail 02 of the surgical table 06. It can be attached to the side rail 02 using a fastener such as a rail clamp 13, more specifically an Allen stirrup clamp. Alternatively, it may be inserted in the optional slots of a side rail 02. The base 12 is dimensioned to be received by the rail clamp 13 or inserted into the slots of a side rail 02. The base 12 can take a variety of shapes, although it is preferably shaped as a rectangular prism in order to more securely mount to the side rail, thus minimizing erratic motion from impacts with the pivotal medical tray 10 during usage in medical procedures. The base 12 has a vertical length suitable to permit the adjustment of the vertical height of the tray assembly 16 above a patient 08, with a preferably vertical length of about 5 inches.

A first end of the shaft 14 is joined to the upper end of the base 12. The shaft 14 is a rigid segment extending upwardly from the base 12 to a second end. The shaft can take a variety of shapes, although it is preferably shaped as a rectangular prism, similar to the preferred shape of the base 12. However, the cross-sectional area of the rectangle presented by the shaft 14 is preferably slightly larger than the cross-sectional area of the rectangle presented by the base 12 in order to present a resting surface when the base 12 is inserted in a side rail 02 having vertically oriented slots. The preferred length of the shaft 14 is about 21 inches.

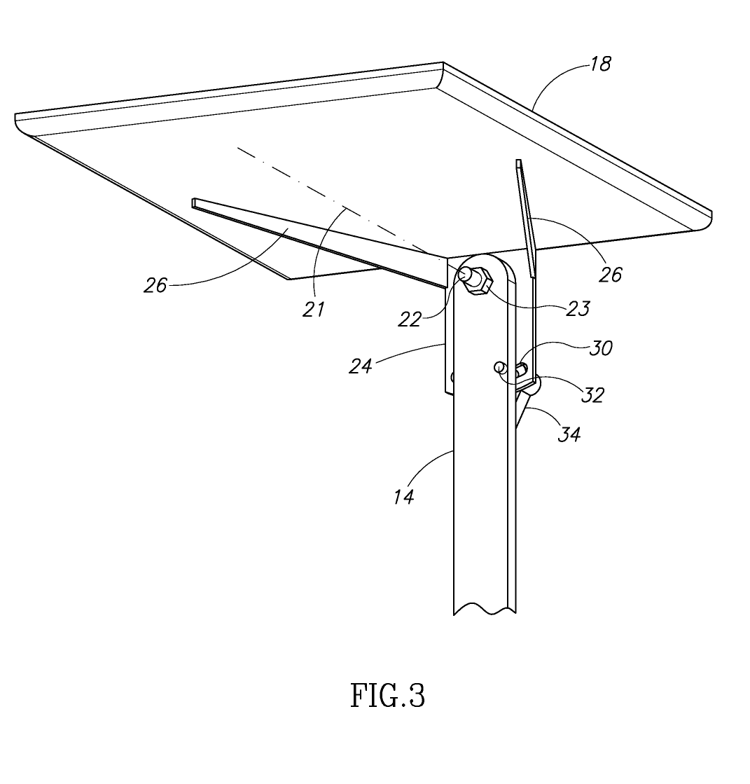

The bracket 24 is comprised of a top edge and a bottom section. The second end of the shaft 14 is rotatably affixed to the bottom section of the bracket 24 using a mechanical fastener 22 23 presenting a pivot point 20. As shown in FIG. 3, the pivot point 20 should be in the range of about ½ inch to about 2 inches below the top edge of the bracket 24, but is preferably about ⅝ inch below the top edge of the bracket 24.

A pivot axis 21 is presented extending orthogonally from the pivot point 20 and laterally along the axis presented by the mechanical fastener 22. The illustrated mechanical faster 22 23 is a bolt and nut pair, with the pivot axis 21 extending along and through the length of the bolt.

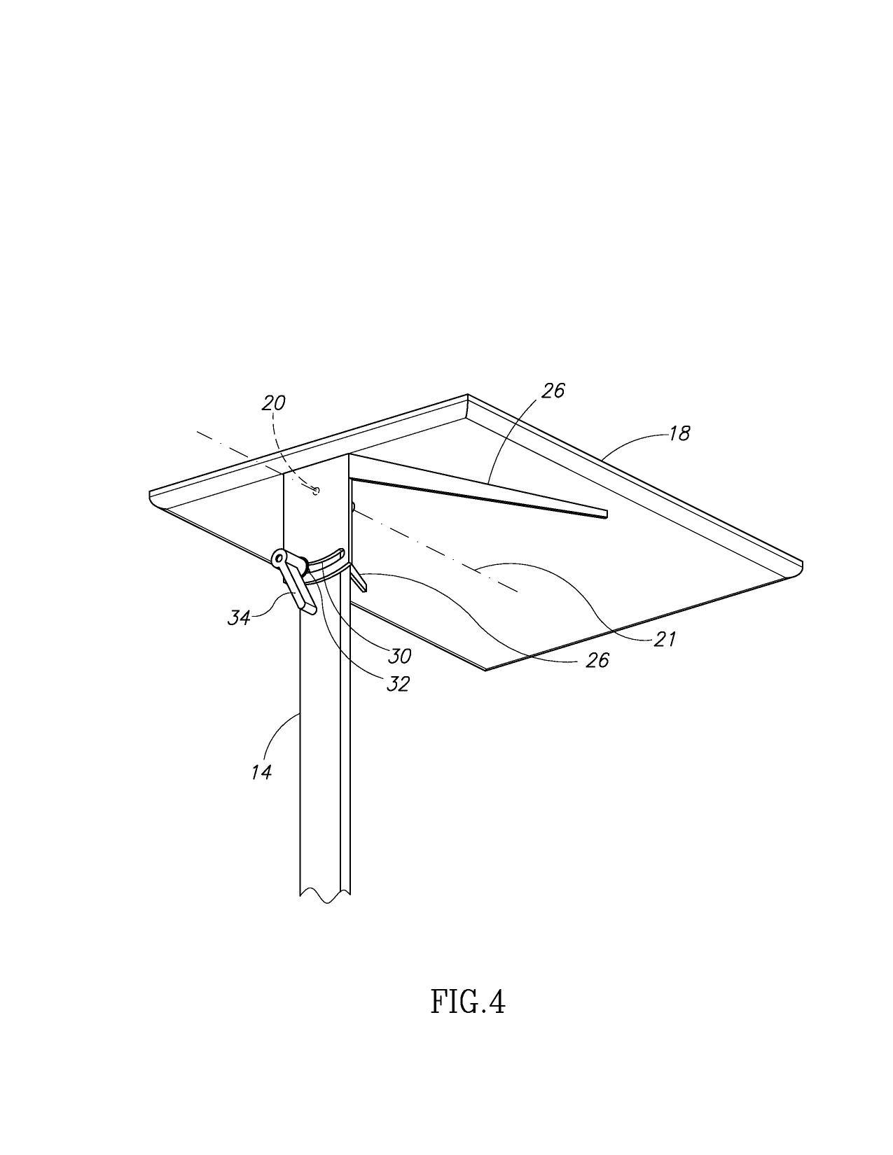

Referring to FIG. 4, the bottom section of the bracket 24 is further comprised of an arcuate slot 30, operable to receive the guide 32. The arcuate slot 30 spans a portion of the width of the bottom section of the bracket 24 at a radial distance ranging from about 1.25 inches to about three inches from the pivot point 20. The arcuate slot 30 is preferably shaped as a circular arc at a radial distance of about 1.75 inches from the pivot point 20. The preferred effective width of the arcuate slot 20 is about 1.75 inches.

A guide 32 is fixed to and protrudes outwardly from the surface of the shaft 14. The guide's 32 width is slightly less than that of the arcuate slot 30 in order to slide therethrough. The guide 32 is optionally fitted with a lock 34, the lock 34 being cooperatively coupled to the guide 32 and providing frictional contact surfaces to selectively secure the pivotal position of the bracket 24 relative to the shaft 24. The depicted guide 32 includes a threaded screw and the depicted lock 34 comprises a hand control and washer. The threaded screw extends outwardly from the shaft 14 through the arcuate slot 30 and through the washer. The head of the lock 34 surrounds and is cooperatively secured to the head of the threaded screw such that the lock 34 and the threaded screw rotate in concert. Upon rotation of the lock 34, the inner surface of the lock 34 causes frictional engagement of the washer to the bottom section of the bracket 24, which in turn causes frictional engagement of the bracket 24 to the shaft 14, securing the angular position of the bracket 24 relative to the shaft 14.

The upper edge of the bracket 24 is joined to the tray assembly 16. In one configuration, the tray assembly 16 includes at least one arm 26 extending substantially horizontally from the top section of the bracket 24, where the arm 26 provides support for a tray 18. In a second configuration, the tray assembly 16 includes a wire frame which defines a perimeter in which a tray 18 may be placed. In yet another configuration, the tray assembly 16 includes at least one arm 26 and a medical tray 18. With the tray assembly 16 joined to the bracket 24, the tray 18 and bracket 24 can cooperatively rotate about the pivot axis 21 throughout the range of the provided arcuate slot 30.

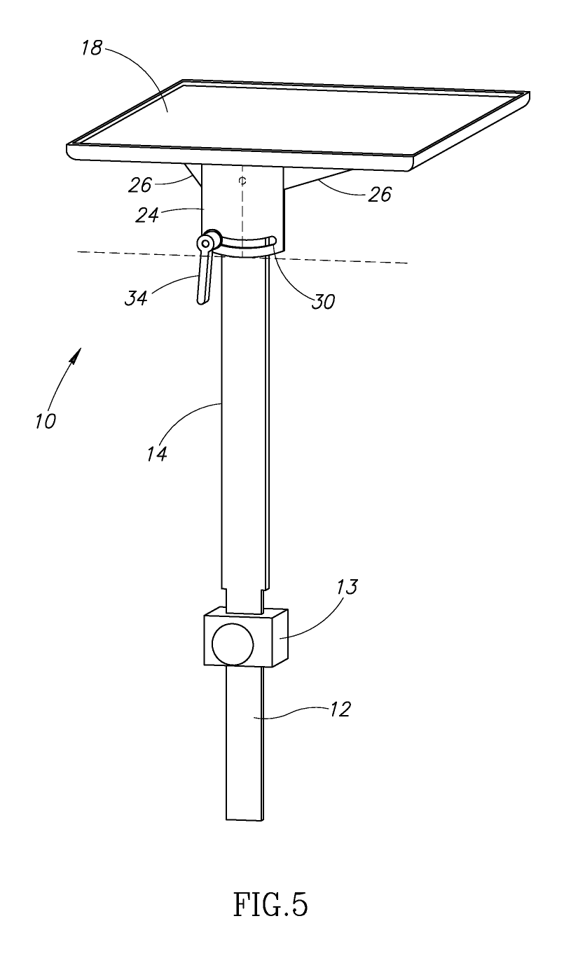

FIG. 5 illustrates an embodiment of the pivotal medical tray 10. It is assembled by joining the base 12 with the first end of the shaft 14. Proximate the second end of the shaft 14, the shaft 14 is joined to the bottom section of the bracket 24 using a mechanical fastener 22 23, defining a pivot point 20. The guide 32 of the shaft is placed through the arcuate slot 30 of the bottom section of the bracket 24. The lock 34 is secured to the guide 32. The tray assembly 16 is joined to the top edge of the bracket 24.

Referring to FIG. 1, in use, the surgical table 06 and the robotic arm assembly 40 are placed in the operating environment. The patient 08 is placed on the surgical table 06. The pivotal medical tray 10 is engaged to the side rail 02 proximate the surgical site and the rail clamp 13 is used after the selected vertical height of the tray is achieved. The lock 34 is released and the tray 18 is manipulated to the desired angular position. The lock 34 is re-engaged to secure the selected angular position of the tray 18. The medical instruments and supplies are placed on the tray 18 and the medical procedure commences.

Insofar as the description above and the accompanying drawing disclose any additional subject matter that is not within the scope of the claims below, the inventions are not dedicated to the public and the right to file one or more applications to claim such additional inventions is reserved.

What is claimed is:

1. A medical tray for attachment to a side rail of a surgical table comprising:
a base;
a shaft having a first end and a second end, said first end joined to said base;
a bracket comprising a top edge and a bottom section;
said shaft extending upwardly and fastened to said bottom section of said bracket proximate said second end of said shaft, defining a pivot point;
said bracket further comprising an arcuate slot in said bottom section;
said shaft further comprising a guide, said guide extending through said arcuate slot; and
a tray assembly joined to said bracket proximate said top edge, whereby said bracket and
said tray assembly cooperatively, selectively rotate about a pivot axis.

2. The device of claim 1 wherein said pivot point is about ½ inch to 2 inches below said top edge of said bracket.

3. The device of claim 1 wherein said pivot point is about ⅝ of an inch below said top edge of said bracket.

4. The device of claim 1 wherein said arcuate slot has a radial distance of about 1.25 inches to 3 inches from said pivot point.

5. The device of claim 1 wherein said arcuate slot has a radial distance of about 1.75 inches from said pivot point.

6. The device of claim 1 wherein said arcuate slot has an effective width of about 1.75 inches.

7. The device of claim 1 wherein said tray assembly comprises at least one arm extending from said top edge of said bracket.

8. The device of claim 1 wherein said tray assembly comprises a tray frame, said tray frame having a perimeter adapted for receipt of a tray.

9. The device of claim 1 wherein said tray assembly comprises a tray and at least one arm extending from said top edge of said bracket.

10. The device of claim 1 further comprising a rail clamp.

11. A medical tray system for attachment to a side rail of a surgical table comprising:
a rectangular prism cross-sectionally shaped base having a length of about 5 inches;
a rectangular prism cross-sectionally shaped shaft having a first end and a second end,
said shaft having a length of about 21 inches, said first end joined to said base;
a bracket comprising a top edge and a bottom section;
said shaft extending upwardly and fastened to said bottom section of said bracket proximate said second end of said shaft, defining a pivot point;
said bracket further comprising an arcuate slot in said bottom section;
said shaft further comprising a guide, said guide extending through said arcuate slot; and
a tray assembly joined to said bracket proximate said top edge, whereby said bracket and
said tray assembly cooperatively selectively rotate about a pivot axis.

12. The device of claim 11 wherein said pivot point is about ½ inch to 2 inches below said top edge of said bracket.

13. The device of claim 11 wherein said pivot point is about ⅝ of an inch below said top edge of said bracket.

14. The device of claim 11 wherein said arcuate slot has a radial distance of about 1.25 inches to 3 inches from said pivot point.

15. The device of claim 11 wherein said arcuate slot has a radial distance of about 1.75 inches from said pivot point.

16. The device of claim 11 wherein said arcuate slot has an effective width of about 1.75 inches.

17. The device of claim 11 wherein said tray assembly comprises at least one arm extending from said top edge of said bracket.

18. The device of claim 11 wherein said tray assembly comprises a tray frame, said tray frame having a perimeter adapted for receipt of a tray.

19. The device of claim 1 wherein said tray assembly comprises a tray and at least one arm extending from said top edge of said bracket.

20. The device of claim 1 further comprising a rail clamp.
